(19) United States
(12) Patent Application Publication
(10) Pub. No.: US 2009/0012051 A1
(43) Pub. Date: Jan. 8, 2009

(54) EXTERNAL PREPARATION

(75) Inventors: Jun Sugishita, Sunto-gun (JP); Yohei Yamazoe, Sunto-gun (JP); Maho Nishikawahara, Shizuoka-shi (JP); Daisuke Harada, Sunto-gun (JP); Katsuya Kobayashi, Sunto-gun (JP); Yoshikazu Tashiro, Mishima-shi (JP)

Correspondence Address: FITZPATRICK CELLA HARPER & SCINTO, 30 ROCKEFELLER PLAZA, NEW YORK, NY, 10112, US

(73) Assignee: KYOWA HAKKO KOGYO CO., LTD., Chiyoda-ku, Tokyo (JP)

(21) Appl. No.: 11/908,232

(22) PCT Filed: Mar. 15, 2006
(86) PCT No.: PCT/JP2006/305096

§ 371 Date: Sep. 10, 2007

(30) Foreign Application Priority Data
Mar. 15, 2005 (JP) 2005-073405

(51) Int. Cl. A61K 31/443; A61K 31/56; A61P 17/00

(52) U.S. Cl. 514/171; 514/278

(57) ABSTRACT

The present invention provides an external preparation which comprises 7-[2-(3,5-dichloro-4-pyridyl)-1-oxoethyl]-4-methoxy-spiro[1,3-benzodioxol-2,1′-cyclopentane] represented by Formula (I) or a pharmaceutically acceptable salt thereof as an active ingredient, comprising 0.5 to 15% by mass of a solvent component in which the solubility of the active ingredient is 4 mg/mL or more, and the like.

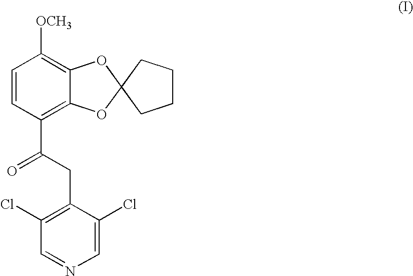

### TECHNICAL FIELD

The present invention relates to an external preparation which comprises 7-[2-(3,5-dichloro-4-pyridyl)-1-oxoethyl]-4-methoxy-spiro[1,3-benzodioxol-2,1′-cyclopentane] or a pharmaceutically acceptable salt thereof.

### BACKGROUND ART

It has been known to use 7-[2-(3,5-dichloro-4-pyridyl)-1-oxoethyl]-4-methoxy-spiro[1,3-benzodioxol-2,1′-cyclopentane] represented by Formula (I):

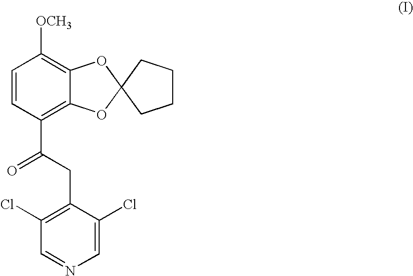

or a pharmaceutically acceptable salt thereof as a phosphodiesterase, IV(PDE-IV) inhibitor (see Patent Document 1).

Furthermore, there are known an agent for treating and/or preventing chronic skin diseases (for example, contact dermatitis, atopic dermatitis, seborrheic dermatitis, nummular eczema, lichen simplex chronicus Vidal, autosensitive dermatitis, stasis dermatitis, asteatotic eczema, psoriasis and the like), comprising 7-[2-(3,5-dichloro-4-pyridyl)-1-oxoethyl]-4-methoxy-spiro[1,3-benzodioxol-2,1′-cyclopentane] or a pharmaceutically acceptable salt thereof as an active ingredient, and an external preparation comprising the compound as an active ingredient (see Patent Document 2).

Meanwhile, in order for an external preparation to effectively manifest its function, it is needed to allow an effective amount of the active ingredient to penetrate into the skin. For this purpose, it is necessary that the external preparation maintains a sufficient amount of the active ingredient, and that the active ingredient is highly released into the skin tissues. As a method for maintaining the active ingredient at high concentration in the external preparation, a method of increasing the solubility of the active ingredient in the external preparation, by incorporating a solvent which has a high solubility for the active ingredient, into the external preparation, is effective (see Non-Patent Document 1). However, if the amount of the incorporated solvent is too much, problems may occur, such as that (i) release of the active ingredient into the skin tissues is decreased, (ii) the skin irritation occurs by the solvent component, and the like. It is also necessary, to keep the quality of the external preparation, that the external preparation contains the active ingredient homogeneously.

Patent Document 1: International Patent Application Publication No. WO 96/36624

Patent Document 2: International Patent Application Publication No. WO 2004/082683

Non-Patent Document 1: International Journal of Pharmaceutics, Vol. 43, p. 31 (1988)

### DISCLOSURE OF THE INVENTION

### Problems to be Solved by the Invention

It is an object of the present invention to provide an external preparation which comprises 7-[2-(3,5-dichloro-4-pyridyl)-1-oxoethyl]-4-methoxy-spiro[1,3-benzodioxol-2,1′-cyclopentane] or a pharmaceutically acceptable salt thereof as an active ingredient, which has excellent homogeneity and excellent releasability of the active ingredient.

### Means for Solving the Problems

The present invention relates to the following items (1) to (43).

(1) An external preparation which comprises 7-[2-(3,5-dichloro-4-pyridyl)-1-oxoethyl]-4-methoxy-spiro[1,3-benzodioxol-2,1′-cyclopentane] represented by Formula (I):

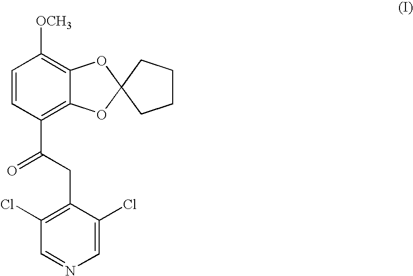

or a pharmaceutically acceptable salt thereof as an active ingredient: comprising 0.5 to 15% by mass of a solvent component in which the solubility of the active ingredient is 4 mg/mL or more.

(2) The external preparation according to the above (1), wherein the solvent component is a solvent component in which the solubility of the active ingredient is 5 mg/mL or more.

(3) The external preparation according to the above (1) or (2), wherein the solvent component is a solvent component selected from the group consisting of propylene carbonate, dipropylene glycol, isopropyl myristate, isopropyl palmitate, diisopropyl sebacate, diethyl sebacate, benzyl alcohol, ethanol and crotamiton.

(4) The external preparation according to the above (1) or (2), wherein the solvent component is propylene carbonate, isopropyl myristate, or a mixed solvent of propylene carbonate and isopropyl myristate.

(5) The external preparation according to any one of the above (1) to (4), wherein the solvent component is contained in an amount of 2 to 10% by mass.

(6) The external preparation according to any one of the above (1) to (5), wherein the active ingredient is contained in an amount of 0.1 to 3% by mass.

(7) The external preparation according to any one of the above (1) to (6), which further comprises a steroid agent.

(8) The external preparation according to the above (7), wherein the steroid agent is a compound selected from the group consisting of clobetasol propionate, diflorasone acetate, betamethasone butyrate propionate, mometasone furancarboxylate, difluprednate, dexamethasone propionate, dexamethasone dipropionate, diflucortolone valerate, fluocinonide, amcinonide, halcinonide, hydrocortisone butyrate propionate, deprodone propionate, dexamethasone valerate, prednisolone valerate acetate, fluocinolone acetonide, hydrocortisone butyrate, alclometasone propionate, triamcinolone acetonide, flumethasone pivalate, clobetasone butyrate, hydrocortisone acetate and prednisolone, or a pharmaceutically acceptable salt thereof.

(9) The external preparation according to any one of the above (1) to (8), which is a therapeutic and/or preventive agent for a disease caused by phosphodiesterase IV hyperfunction.

(10) The external: preparation according to the above (9), wherein the disease caused by phosphodiesterase IV hyperfunction is a chronic skin disease.

(11) The external preparation according to the above (10), wherein the chronic skin disease is a disease selected from the group consisting of contact dermatitis, atopic dermatitis, seborrheic dermatitis, nummular eczema, lichen simplex chronicus Vidal, autosensitive dermatitis, stasis dermatitis, asteatotic eczema and psoriasis.

(12) A method for homogenizing an active ingredient in an external preparation which comprises 7-[2-(3,5-dichloro-4-pyridyl)-1-oxoethyl]-4-methoxy-spiro[1,3-benzodioxol-2,1′-cyclopentane] represented by Formula (I):

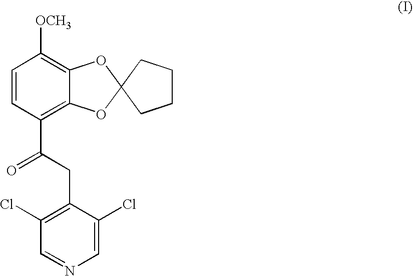

or a pharmaceutically acceptable salt thereof as an active ingredient, comprising allowing a solvent component in which the solubility of the active ingredient is 4 mg/mL or more to exist in the external preparation in an amount of 0.5 to 15% by mass.

(13) The method according to the above (12), wherein the solvent component is a solvent component in which the solubility of the active ingredient is 5 mg/mL or more.

(14) The method according to the above (12) or (13), wherein the solvent component is a solvent component selected from the group consisting of propylene carbonate, dipropylene glycol, isopropyl myristate, isopropyl palmitate, diisopropyl sebacate, diethyl sebacate, benzyl alcohol, ethanol and crotamiton.

(15) The method according to the above (12) or (13), wherein the solvent component is propylene carbonate, isopropyl myristate, or a mixed solvent of propylene carbonate and isopropyl myristate.

(16) The method according to any one of the above (12) to (15), wherein the solvent component is allowed to exist in the external preparation in an amount of 2 to 10% by mass.

(17) The method according to any one of the above (12) to (16), wherein the external preparation is an external preparation wherein the active ingredient is contained in an amount of 0.1 to 3% by mass.

(18) The method according to any one of the above (12) to (17), wherein the external preparation further comprises a steroid agent.

(19) The method according to the above (18), wherein the steroid agent is a compound selected from the group consisting of clobetasol propionate, diflorasone acetate, betamethasone butyrate propionate, mometasone furancarboxylate, difluprednate, dexamethasone propionate, dexamethasone dipropionate, diflucortolone valerate, fluocinonide, amcinonide, halcinonide, hydrocortisone butyrate propionate, deprodone propionate, dexamethasone valerate, prednisolone valerate acetate, fluocinolone acetonide, hydrocortisone butyrate, alclometasone propionate, triamcinolone acetonide, flumethasone pivalate, clobetasone butyrate, hydrocortisone acetate and prednisolone, or a pharmaceutically acceptable salt thereof.

(20) A method for enhancing the release of an active ingredient from an external preparation which comprises 7-[2-(3,5-dichloro-4-pyridyl)-1-oxoethyl]-4-methoxy-spiro[1,3-benzodioxol-2,1′-cyclopentane] represented by Formula (I):

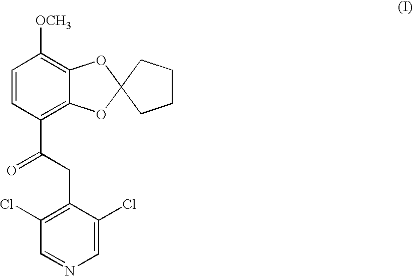

or a pharmaceutically acceptable salt thereof as an active ingredient, comprising allowing a solvent component in which the solubility of the active ingredient is 4 mg/mL or more to exist in the external preparation in an amount of 0.5 to 15% by mass.

(21) The method according to the above (20), wherein the solvent component is a solvent component in which the solubility of the active ingredient is 5 mg/mL or more.

(22) The method according to the above (20) or (21), wherein the solvent component is a solvent component selected from the group consisting of propylene carbonate, dipropylene glycol, isopropyl myristate, isopropyl palmitate, diisopropyl sebacate, diethyl sebacate, benzyl alcohol, ethanol and crotamiton.

(23) The method according to the above (20) or (21), wherein the solvent component is propylene carbonate, isopropyl myristate, or a mixed solvent of propylene carbonate and isopropyl myristate.

(24) The method according to any one of the above (20) to (23), wherein the solvent component is allowed to exist in the external preparation in an amount of 2 to 10% by mass.

(25) The method according to any one of the above (20) to (24), wherein the external preparation is an external preparation wherein the active ingredient is contained in an amount of 0.1 to 3% by mass.

(26) The method according to any one of the above (20) to (25), wherein the external preparation further comprises a steroid agent.

(27) The method according to the above (26), wherein the steroid agent is a compound selected from the group consisting of clobetasol propionate, diflorasone acetate, betamethasone butyrate propionate, mometasone furancarboxylate, difluprednate, dexamethasone propionate, dexamethasone dipropionate, diflucortolone valerate, fluocinonide, amcinonide, halcinonide, hydrocortisone butyrate propionate, deprodone propionate, dexamethasone valerate, prednisolone valerate acetate, fluocinolone acetonide, hydrocortisone butyrate, alclometasone propionate, triamcinolone acetonide, flumethasone pivalate, clobetasone butyrate, hydrocortisone acetate and prednisolone, or a pharmaceutically acceptable salt thereof.

(28) Use of an external preparation which comprises 7-[2-(3,5-dichloro-4-pyridyl)-1-oxoethyl]-4-methoxy-spiro[1,3-benzodioxol-2,1′-cyclopentane] represented by Formula (I):

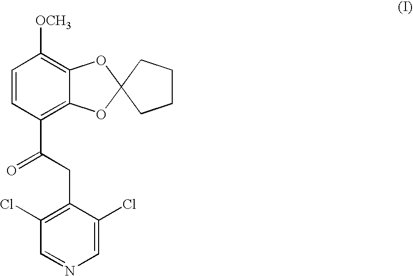

or a pharmaceutically acceptable salt thereof as an active ingredient for homogenizing the active ingredient, wherein the external preparation contains a solvent component in which the solubility of the active ingredient is 4 mg/mL or more in an amount of 0.5 to 15% by mass.

(29) The use according to the above (28), wherein the solvent component is a solvent component in which the solubility of the active ingredient is 5 mg/mL or more.

(30) The use according to the above (28) or (29), wherein the solvent component is a solvent component selected from the group consisting of propylene carbonate, dipropylene glycol, isopropyl myristate, isopropyl palmitate, diisopropyl sebacate, diethyl sebacate, benzyl alcohol, ethanol and crotamiton.

(31) The use according to the above (28) or (29), wherein the solvent component is propylene carbonate, isopropyl myristate, or a mixed solvent of propylene carbonate and isopropyl myristate.

(32) The use according to any one of the above (28) to (31), wherein the solvent component is allowed to exist in the external preparation in an amount of 2 to 10% by mass.

(33) The use according to any one of the above (28) to (32), wherein the external preparation is an external preparation comprising the active ingredient in an amount of 0.1 to 3% by mass.

(34) The use according to any one of the above (28) to (33), wherein the external preparation is an external preparation which further comprises a steroid agent.

(35) The use according to the above (34), wherein the steroid agent is a compound selected from the group consisting of clobetasol propionate, diflorasone acetate, betamethasone butyrate propionate, mometasone furancarboxylate, difluprednate, dexamethasone propionate, dexamethasone dipropionate, diflucortolone valerate, fluocinonide, amcinonide, halcinonide, hydrocortisone butyrate propionate, deprodone propionate, dexamethasone valerate, prednisolone valerate acetate, fluocinolone acetonide, hydrocortisone butyrate, alclometasone dipropionate, triamcinolone acetonide, flumethasone pivalate, clobetasone butyrate, hydrocortisone acetate and prednisolone, or a pharmaceutically acceptable salt thereof.

(36) Use of an external preparation which comprises 7-[2-(3,5-dichloro-4-pyridyl)-1-oxoethyl]-4-methoxy-spiro[1,3-benzodioxol-2,1′-cyclopentane] represented by Formula (I):

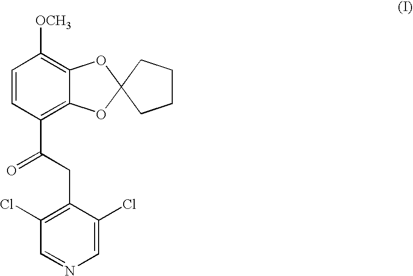

or a pharmaceutically acceptable salt thereof as an active ingredient for enhancing the release of the active ingredient from the external preparation, wherein the external preparation contains a solvent component in which the solubility of the active ingredient is 4 mg/mL or more in an amount of 0.5 to 15% by mass.

(37) The use according to the above (36), wherein the solvent component is a solvent component in which the solubility of the active ingredient is 5 mg/mL or more.

(38) The use according to the above (36) or (37), wherein the solvent component is a solvent component selected from the group consisting of propylene carbonate, dipropylene glycol, isopropyl myristate, isopropyl palmitate, diisopropyl sebacate, diethyl sebacate, benzyl alcohol, ethanol and crotamiton.

(39) The use according to the above (36) or (37), wherein the solvent component is propylene carbonate, isopropyl myristate, or a mixed solvent of propylene carbonate and isopropyl myristate.

(40) The use according to any one of the above (36) to (39), wherein the solvent component is allowed to exist in the external preparation in an amount of 2 to 10% by mass.

(41) The use according to any one of the above (36) to (40), wherein the external preparation is an external preparation comprising the active ingredient in an amount of 0.1 to 3% by mass.

(42) The use according to any one of the above (36) to (41), wherein the external preparation is an external preparation which further comprises a steroid agent.

(43) The use according to the above (42), wherein the steroid agent is a compound selected from the group consisting of clobetasol propionate, diflorasone acetate, betamethasone butyrate propionate, mometasone furancarboxylate, difluprednate, dexamethasone propionate, dexamethasone dipropionate, diflucortolone valerate, fluocinonide, amcinonide, halcinonide, hydrocortisone butyrate propionate, deprodone propionate, dexamethasone valerate, prednisolone valerate acetate, fluocinolone acetonide, hydrocortisone butyrate, alclometasone propionate, triamcinolone acetonide, flumethasone pivalate, clobetasone butyrate, hydrocortisone acetate and prednisolone, or a pharmaceutically acceptable salt thereof.

### EFFECTS OF THE INVENTION

According to the present invention, there can be provided an external preparation which has excellent homogeneity and excellent releasability of the active ingredient, and the like wherein the external preparation comprises 7-[2-(3,5-dichloro-4-pyridyl)-1-oxoethyl]-4-methoxy-spiro[1,3-benzodioxol-2,1′-cyclopentane] or a pharmaceutically acceptable salt thereof, which is useful for various diseases caused by PDE-IV hyperfunction [for example, a chronic skin disease (for example, contact dermatitis, atopic dermatitis, seborrheic dermatitis, nummular eczema, Lichen simplex chronicus Vidal, autosensitive dermatitis, stasis dermatitis, asteatotic eczema, psoriasis, etc.), asthma, COPD, allergic diseases or the like], as an active ingredient.

### BEST MODE FOR CARRYING OUT THE INVENTION

Hereinafter, 7-[2-(3,5-dichloro-4-pyridyl)-1-oxoethyl]-4-methoxy-spiro[1,3-benzodioxol-2,1′-cyclopentane] represented by Formula (I) is referred to as Compound (I).

The pharmaceutically acceptable salts of the Compound (I) include acid addition salts, metal salts, ammonium salts, organic amine addition salts, amino acid addition salts and the like, that are pharmaceutically acceptable.

The pharmaceutically acceptable acid addition salts of Compound (I) include, for example, inorganic acid salts such as hydrochloride, sulfate, nitrate, and phosphate; and organic acid salts such as acetate, maleate, fumarate, and citrate. The pharmaceutically acceptable metal salts include, for example, alkali metal salts such as sodium salt and potassium salt; alkaline earth metal salts such as magnesium salt and calcium salt; aluminum salt; zinc salt and the like. The pharmaceutically acceptable ammonium salts include, for example, salts of ammonium, tetramethylammonium or the like. The pharmaceutically acceptable organic amine addition salts include, for example, addition salts of morpholine, piperidine or the like. The pharmaceutically acceptable amino acid addition salts include, for example, addition salts of glycine, phenylalanine, lysine, aspartic acid, glutamic acid or the like.

Next, a method for producing Compound (I) are described below.

Compound (I) can be produced by the method described in WO 96/36624.

Compound (I) can exist as tautomers and the like, however, the external preparation of the present invention can use all possible isomers including the above-mentioned isomers, as well as the mixtures thereof.

To obtain a salt of Compound (I), when Compound (I) is obtained in the form of the salt, it may be purified as it is. Further, when compound (I) is obtained in a free form, Compound (I) may be dissolved or suspended in a suitable solvent, followed by addition of an acid or a base to form a salt. Then, the resulting salt may be isolated and purified.

Compound (I) and a pharmaceutically acceptable salts thereof may exist in the form of adducts with water or various solvents, but such adducts also can be used for the external preparation of the present invention.

The external preparation of the present invention is a pharmaceutical preparation comprising Compound (I) or a pharmaceutically acceptable salt thereof, and a solvent component, and is prepared by mixing the Compound (I) or a pharmaceutically acceptable salt thereof and the solvent component, with one or more pharmaceutically acceptable carriers, and then subjecting the mixture to any method well known in the technical field of pharmaceutics.

The content of Compound (I) or a pharmaceutically acceptable salt thereof in the external preparation of the present invention is preferably 1 to 100 mg (0.1 to 10% by mass), more preferably 1 to 50 mg (0.1 to 5% by mass), even more preferably 1 to 30 mg (0.1 to 3% by mass), and still more preferably 3 to 10 mg (0.3 to 1% by weight) per 1 g of the external preparation.

The solvent component is not particularly limited as long as it is a solvent in which the solubility of Compound (I) or a pharmaceutically acceptable salt thereof is 4 mg/mL or more, preferably 5 mg/mL or more, and more preferably 10 mg/mL or more, and which is pharmaceutically acceptable in an external preparation. Specific examples thereof include propylene carbonate, dipropylene glycol, isopropyl myristate, isopropyl palmitate, diisopropyl sebacate, diethyl sebacate, benzyl alcohol, ethanol, crotamiton and the like, and preferred examples include propylene carbonate, isopropyl palmitate, isopropyl myristate and the like. These can be used alone or as a mixture.

The content of the solvent component in the external preparation of the present invention is 0.5 to 15% by mass, preferably 1 to 10% by mass, more preferably 2 to 10% by mass, and even more preferably 2 to 5% by weight. If the content of the solvent component is too less, the solvent component cannot sufficiently dissolve Compound (I) or a pharmaceutically acceptable salt thereof in the preparation, and it is difficult to homogeneously disperse and maintain a desired amount of the Compound (I) or a pharmaceutically acceptable salt thereof in the external preparation. Further, if the content of the solvent component is too much, Compound (I) or a pharmaceutically acceptable salt thereof may not release sufficiently, or skin irritation caused by the solvent component may occur.

Dosage form that is suitable for the external preparation of the present invention is not particularly limited, and include any of a formulation in the form of cream, paste, jelly, gel, emulsion, liquid or the like (ointment, cream, liniment, lotion, etc.); a formulation prepared by dissolving or mixing and dispersing the active ingredient and a percutaneous absorption enhancer, and spreading the mixture on backing sheet (patches, etc.); a formulation prepared by dissolving or mixing and dispersing the active ingredient and a percutaneous absorption enhancer in an adhesive, and spreading the mixture on backing sheet (plaster, tape, etc.); and the like. A preparation in the form of ointment, cream or lotion is preferred, and an ointment is more preferred. As for the base of each dosage forms described above, any material can be used as long as it is pharmaceutically acceptable. As the base for ointment, cream, liniment, lotion and the like, any commonly known material can be used, and examples thereof include sodium alginate; gelatin; corn starch; tragacanth gum; methylcellulose; hydroxyethyl cellulose; carboxymethyl cellulose; xanthan gum; dextrin; carboxymethyl starch; polymers such as polyvinyl alcohol, sodium polyacrylate, methoxyethylene-maleic anhydride copolymer, polyvinyl ether and polyvinylpyrrolidone; oils and fats such as yellow beeswax, bleached beeswax, olive oil, cacao oil, sesame oil, soybean oil, camellia oil, peanut oil, beef tallow, lard, and lanolin; white Vaseline; yellow Vaseline; paraffin; gelated hydrocarbons; higher fatty acids such as stearic acid and the like; higher alcohols such as cetyl alcohol and stearyl alcohol; polyethylene glycol; water; and the like. White Vaseline and the like are preferred. Furthermore, if necessary, inorganic fillers such as kaolin, bentonite, zinc oxide and titanium oxide; viscosity controlling agents; anti-aging agents; pH adjusting agents; moisturizers such as glycerin and propylene glycol; solubilizing agents such as ethylene carbonate; surfactants such as polyoxyethylene hydrogenated castor oil, polyoxyethylene sorbitan fatty acid esters, polyoxyethylene alkyl ethers, polyoxyethylene alkyl ether phosphoric acid esters, glycerin fatty acid esters, sorbitan fatty acid esters, sucrose fatty acid esters, fatty acid soaps, alkylsulfosuccinate, alkylsulfate, alkylamine salts, alkyl quaternary ammonium salts and alkylpyridinium salts; and the like may also be added.

The external preparation can also contain one or more additives selected from diluents, flavors, excipients, disintegrants, lubricants, binders, plasticizers, preservatives and the like.

The external preparation of the present invention can also contain an active ingredient for any other therapy, in addition to the Compound (I) or a pharmaceutically acceptable salt thereof, as the active ingredient.

Examples of the active ingredient for any other therapy include, for example, steroid agents, immunosuppressants, anti-inflammatory agents such as cyclooxygenase inhibitors, anti-allergic agents such as anti-histamine agents, leukotriene antagonists, vitamin D derivatives, retinoid external preparations, antimicrobial agents, antibiotics, and the like. Preferably, steroid agents, immunosuppressants and vitamin D derivatives may be included.

As the steroid agent, any steroid agent can be used so long as it reduces factors caused by inflammatory response such as cytokines or the number of mast cells and eosinophils, and suppresses the migration or the activation of inflammatory cells. Examples thereof include betamethasones such as betamethasone valerate, betamethasone butyrate propionate and betamethasone dipropionate, dexamethasones such as dexamethasone propionate, dexamethasone valerate and dexamethasone dipropionate, hydrocortisones such as hydrocortisone, hydrocortisone valerate, hydrocortisone butyrate, hydrocortisone acetate and hydrocortisone butyrate propionate, prednisolones such as prednisolone and prednisolone valerate acetate, clobetasol propionate, diflorasone acetate, mometasone furancarboxylate, difluprednate, diflucortolone valerate, fluocinonide, amcinonide, halcinonide, deprodone propionate, fluocinolone acetonide, triamcinolone acetonide, alclometasone propionate, flumethasone pivalate, clobetasone butyrate, halobetasol propionate, desoxymethasone, fluticasone propionate, flurandrenolide, desonide, alclometasone dipropionate, flumethasone pivalate and the like. Preferred examples thereof include betamethasone butyrate propionate, dexamethasone propionate, dexamethasone valerate, dexamethasone dipropionate, hydrocortisone butyrate, hydrocortisone acetate, hydrocortisone butyrate propionate, clobetasol propionate, diflorasone acetate, mometasone furancarboxylate, difluprednate, diflucortolone valerate, fluocinonide, amcinonide, halcinonide, deprodone propionate, prednisolone, prednisolone valerate acetate, fluocinolone acetonide, triamcinolone acetonide, alclometasone propionate, flumethasone pivalate, clobetasone butyrate and the like. Further, these compounds may be used alone or in combination.

The dose ratio (weight/weight) of Compound (I) or a pharmaceutically acceptable salt thereof to the steroid agent may be appropriately adjusted in accordance with the type, efficacy and the like of the steroid agent used, and specifically, the dose ratio is, for example, 1/50 (Compound (I) or a pharmaceutically acceptable salt/the steroid agent) to 50000/1, preferably 1/30 to 10000/1, more preferably 1/20 to 5000/1, and even more preferably 1/10 to 1000/1.

As the immunosuppressant, any immunosuppressant can be used so long as it reduces factors caused by inflammatory response such as cytokines or the number of mast cells and eosinophils, and suppresses the migration or the activation of inflammatory cells. Examples thereof include, Tacrolimus, Pimecrolimus, Ascomycin, Rapamycin, FTY 720, Azathioprine, Cyclophosphamide, Mizoribin, Methotrexate, Cyclosporin A, Mofetil mycophenolate, Brequinar sodium, Deoxyspergualin, Leflunomide and the like. Among them, Tacrolimus, Pimecrolimus, Ascomycin, Rapamycin, FTY 720, Azathioprine, Cyclophosphamide, Mizoribin, Methotrexate, Cyclosporin A and the like are preferred. These may be used alone or in combination.

The dose ratio (weight/weight) of Compound (I) or a pharmaceutically acceptable salt thereof to the immunosuppressant may be appropriately adjusted in accordance with the type, efficacy and the like of the immunosuppressant used, and specifically, the dose ratio is, for example, 1/50 (Compound (I) or a pharmaceutically acceptable salt/the immunosuppressant) to 50000/1, preferably 1/30 to 10000/1, more preferably 1/20 to 5000/1, and even more preferably 1/10 to 1000/1.

The vitamin D derivative includes, for example, Vitamin D2, Vitamin D3, Calcitriol, Calcipotriol, Tacalcitol, MC01288, CB1093, Falecalcitriol, Lexacalcitriol, Maxacalcitriol, Seocalcitriol, EB-1213, EL-715, GS-1500, KH-1230, KH-1266, LR-103, Paricalcitriol, and the like.

The dose ratio (weight/weight) of Compound (I) or a pharmaceutically acceptable salt thereof to the vitamin D derivative may be appropriately adjusted in accordance with the type, efficacy and the like of the vitamin D derivative used, and specifically, the dose ratio is, for example, 1/50 (Compound (I) or a pharmaceutically acceptable salt/the vitamin D derivative) to 50000/1, preferably 1/30 to 10000/1, more preferably 1/20 to 5000/1, and even more preferably 1/10 to 1000/1.

The term “homogeneous” as used in the method for homogenizing an active ingredient of the present invention means that Compound (I) or a pharmaceutically acceptable salt thereof is homogenously maintained in the external preparation without precipitating or separating. According to the method for homogenizing an active ingredient of the present invention, it is possible to homogeneously maintain Compound (I) or a pharmaceutically acceptable salt thereof in the external preparation, for example, at a concentration sufficient for exhibiting the efficacy, preferably at a concentration of 0.1 to 10% by mass, more preferably 0.1 to 5% by mass, even more preferably 0.1 to 3% by mass, and still more preferably 0.3 to 1% by mass.

Furthermore, the term “enhancing” as used in the method for enhancing the release of an active ingredient from an external preparation of the present invention means that when an external preparation containing Compound (I) or a pharmaceutically acceptable salt thereof is applied to the skin, the amount of Compound (I) or a pharmaceutically acceptable salt thereof released from the external preparation onto the skin is increased. According to the method for enhancing the release of an active ingredient of the present invention, it is possible to increase the amount of Compound (I) or a pharmaceutically acceptable salt thereof released onto the skin. For example, in the case of applying an external preparation containing Compound (I) or a pharmaceutically acceptable salt thereof on the skin, the external preparation of the present invention can release the active ingredient in an amount sufficient for exhibiting the efficacy.

The doses and the dosage frequency of Compound (I) or a pharmaceutically acceptable salt thereof may vary with age and body weight of a patient, nature or severity of the symptom to be treated, and the like, but the Compound (I) or a pharmaceutically acceptable salt thereof is usually administered once or several times a day, by applying onto the skin. A single dose of Compound (I) or a pharmaceutically acceptable salt thereof is preferably 0.01 to 30 mg, and more preferably 0.03 to 10 mg. However, these doses and dosage frequencies vary with depending upon various conditions described above.

Hereinafter, embodiments of the present invention will be described with reference to Comparative Examples, Examples and Test Examples, but the scope of the present invention is not to be limited by these examples.

### COMPARATIVE EXAMPLE 1

An external preparation including the following composition was prepared according to the conventional manner. Compound (I) (60 mg) was added to white Vaseline (1940 mg) with heating and stirring, and the Compound (I) was dispersed therein with continuously stirring and heating. Subsequently, the dispersion was left to stand at room temperature (about 25° C.) to obtain the external ointment.

TABLE 1

| | | |
|---|---|---|
| Prescription | Compound (I) | 60 mg |
| | White vaseline | 1940 mg |
| | | 2000 mg |

The obtained ointment was observed under a microscope, and as a result, the preparation which did not contain a solvent component was found to show precipitation of Compound (I), and did not maintain the homogeneity in the preparation.

### COMPARATIVE EXAMPLE 2

An external preparation including the following composition was prepared according to the conventional manner. Bleached beeswax (350 mg), propylene carbonate (solubility of Compound (I): about 33 mg/mL) (1000 mg), ethylene carbonate (150 mg), isopropyl myristate (solubility of Compound (I): about 10 mg/mL) (1750 mg), stearyl alcohol (100 mg) and sorbitan monostearate (100 mg) were added to white Vaseline (1535 mg) with heating and stirring, and dispersed therein. Compound (I) (15 mg) was added to the dispersion, and the resulting mixture was heated with continuously stirring. Subsequently, the mixture was dispersed by a high speed homogenizer with heating, and the dispersion was left to stand at room temperature (about 25° C.) to obtain the external ointment.

TABLE 2

| | | |
|---|---|---|
| Prescription | Compound (I) | 15 mg |
| | White vaseline | 1535 mg |
| | Bleached beeswax | 350 mg |
| | Propylene carbonate | 1000 mg |
| | Ethylene carbonate | 150 mg |
| | Isopropyl myristate | 1750 mg |
| | Stearyl alcohol | 100 mg |
| | Sorbitan monostearate | 100 mg |
| | | 5000 mg |

The obtained ointment was observed under a microscope, and as a result, the preparation which contained more than 15% by mass of solvent components was not found to show precipitation of Compound (I), and maintained the homogeneity in the preparation.

### COMPARATIVE EXAMPLE 3

An external preparation including the following composition was prepared according to the conventional manner. Bleached beeswax (350 mg), propylene carbonate (solubility of Compound (I): about 33 mg/mL) (1000 mg), ethylene carbonate (150 mg), isopropyl myristate (solubility of Compound (I): about 10 mg/mL) (1750 mg), stearyl alcohol (100 mg) and sorbitan monostearate (100 mg) were added to the white Vaseline (1500 mg) with heating and stirring, and dispersed therein. Compound (I) (50 mg) was added to the dispersion, and the resulting mixture was heated with continuously stirring. Subsequently, the mixture was dispersed by a high speed homogenizer with heating, and the dispersion was left to stand at room temperature (about 25° C.) to obtain the external ointment.

TABLE 3

| | | |
|---|---|---|
| Prescription | Compound (I) | 50 mg |
| | White vaseline | 1500 mg |
| | Bleached beeswax | 350 mg |
| | Propylene carbonate | 1000 mg |
| | Ethylene carbonate | 150 mg |
| | Isopropyl myristate | 1750 mg |
| | Stearyl alcohol | 100 mg |
| | Sorbitan monostearate | 100 mg |
| | | 5000 mg |

The obtained ointment was observed under a microscope, and as a result, the preparation which contained more than 15% by mass of solvent components was not found to show precipitation of Compound (I), and maintained the homogeneity in the preparation.

### COMPARATIVE EXAMPLE 4

An external preparation including the following composition was prepared according to the conventional manner. Bleached beeswax (350 mg), propylene carbonate (solubility of Compound (I): about 33 mg/mL) (1000 mg), ethylene carbonate (150 mg), isopropyl myristate (solubility of Compound (I): about 10 mg/mL) (1750 mg), stearyl alcohol (100 mg) and sorbitan monostearate (100 mg) were added to the white Vaseline (1400 mg) with heating and stirring, and dispersed therein. Compound (I) (150 mg) was added to the dispersion, and the resulting mixture was heated with continuously stirring. Subsequently, the mixture was dispersed by a high speed homogenizer with heating, and the dispersion was left to stand at room temperature (about 25° C.) to obtain the external ointment.

TABLE 4

| | | |
|---|---|---|
| Prescription | Compound (I) | 150 mg |
| | White vaseline | 1400 mg |
| | Bleached beeswax | 350 mg |
| | Propylene carbonate | 1000 mg |
| | Ethylene carbonate | 150 mg |
| | Isopropyl myristate | 1750 mg |
| | Stearyl alcohol | 100 mg |
| | Sorbitan monostearate | 100 mg |
| | | 5000 mg |

The obtained ointment was observed under a microscope, and as a result, the preparation which contained more than 15% by mass of solvent components was not found to show precipitation of Compound (I), and maintained the homogeneity in the preparation.

### COMPARATIVE EXAMPLE 5

An external preparation including the following composition was prepared according to the conventional manner. Bleached beeswax (160 mg) and isopropyl myristate (solubility of Compound (I): about 10 mg/mL) (380 mg) were added to the white Vaseline (1440 mg) with heating and stirring, and dispersed therein. Compound (I) (20 mg) was added to the dispersion, and the resulting mixture was heated with continuously stirring. Subsequently, the mixture was left to stand at room temperature (about 25° C.) to obtain the external ointment.

TABLE 5

| | | |
|---|---|---|
| Prescription | Compound (I) | 20 mg |
| | White vaseline | 1440 mg |
| | Bleached beeswax | 160 mg |
| | Isopropyl myristate | 380 mg |
| | | 2000 mg |

The obtained ointment was observed under a microscope, and as a result, the preparation which contained more than 15% by mass of a solvent component was not found to show precipitation of Compound (I), and maintained the homogeneity in the preparation.

### COMPARATIVE EXAMPLE 6

An external preparation including the following composition was prepared according to the conventional manner. Propylene glycol (solubility of Compound (I): about 3 mg/mL) (500 mg) and cetyl octanoate (100 mg) were added to the white Vaseline (1340 mg) with heating and stirring, and dispersed therein. Compound (I) (60 mg) was added to the dispersion, and the resulting mixture was heated with continuously stirring. Subsequently, the mixture was left to stand at room temperature (about 25° C.) to obtain the external ointment.

TABLE 6

| | | |
|---|---|---|
| Prescription | Compound (I) | 60 mg |
| | White vaseline | 1340 mg |
| | Propylene glycol | 500 mg |
| | Cetyl octanoate | 100 mg |
| | | 2000 mg |

The obtained ointment was observed under a microscope, and as a result, the preparation which contained a solvent component in which the solubility of Compound (I) was less than 4 mg/mL, was found to show precipitation of Compound (I), and did not maintain the homogeneity in the preparation.

### EXAMPLE 1

An external preparation including the following composition was prepared according to the conventional manner. Bleached beeswax (160 mg), liquid paraffin (340 mg) and propylene carbonate (solubility of Compound (I): about 33 mg/mL) (40 mg) were added to the white Vaseline (1454 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 7

| | | |
|---|---|---|
| Prescription | Compound (I) | 6 mg |
| | White vaseline | 1454 mg |
| | Bleached beeswax | 160 mg |
| | Liquid paraffin | 340 mg |
| | Propylene carbonate | 40 mg |
| | | 2000 mg |

The obtained ointment was observed under a microscope, and as a result, the preparation was not found to show precipitation of Compound (I), and maintained the homogeneity in the preparation.

### EXAMPLE 2

An external preparation including the following composition was prepared according to the conventional manner. Bleached beeswax (160 mg), liquid paraffin (280 mg) and propylene carbonate (solubility of Compound (I): about 33 mg/mL) (100 mg) were added to the white Vaseline (1454 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 8

| | | |
|---|---|---|
| Prescription | Compound (I) | 6 mg |
| | White vaseline | 1454 mg |
| | Bleached beeswax | 160 mg |
| | Liquid paraffin | 280 mg |
| | Propylene carbonate | 100 mg |
| | | 2000 mg |

The obtained ointment was observed under a microscope, and as a result, the preparation was not found to show precipitation of Compound (I), and maintained the homogeneity in the preparation.

### EXAMPLE 3

An external preparation including the following composition was prepared according to the conventional manner. Bleached beeswax (160 mg), liquid paraffin (280 mg) and propylene carbonate (solubility of Compound (I): about 33 mg/mL) (100 mg) were added to the white Vaseline (1440 mg) with heating and stirring, and dispersed therein. Compound (I) (20 mg) was added to the dispersion, and the resulting mixture was heated with continuously stirring. Subsequently, the mixture was left to stand at room temperature (about 25° C.) to obtain the external ointment.

TABLE 9

| | | |
|---|---|---|
| Prescription | Compound (I) | 20 mg |
| | White vaseline | 1440 mg |
| | Bleached beeswax | 160 mg |
| | Liquid paraffin | 280 mg |
| | Propylene carbonate | 100 mg |
| | | 2000 mg |

The obtained ointment was observed under a microscope, and as a result, the preparation was not found to show precipitation of Compound (I), and maintained the homogeneity in the preparation.

### EXAMPLE 4

An external preparation including the following composition was prepared according to the conventional manner. Bleached beeswax (160 mg), liquid paraffin (340 mg) and benzyl alcohol (solubility of Compound (I): about 100 mg/mL) (40 mg) were added to the white Vaseline (1454 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 10

| | | |
|---|---|---|
| Prescription | Compound (I) | 6 mg |
| | White vaseline | 1454 mg |
| | Bleached beeswax | 160 mg |
| | Liquid paraffin | 340 mg |
| | Benzyl alcohol | 40 mg |
| | | 2000 mg |

The obtained ointment was observed under a microscope, and as a result, the preparation was not found to show precipitation of Compound (I), and maintained the homogeneity in the preparation.

### EXAMPLE 5

An external preparation including the following composition was prepared according to the conventional manner. Bleached beeswax (160 mg), liquid paraffin (340 mg) and benzyl alcohol (solubility of Compound (I): about 100 mg/mL) (40 mg) were added to the white Vaseline (1440 mg) with heating and stirring, and dispersed therein. Compound (I) (20 mg) was added to the dispersion, and the resulting mixture was heated with continuously stirring. Subsequently, the mixture was left to stand at room temperature (about 25° C.) to obtain the external ointment.

TABLE 11

| | | |
|---|---|---|
| Prescription | Compound (I) | 20 mg |
| | White vaseline | 1440 mg |
| | Bleached beeswax | 160 mg |
| | Liquid paraffin | 340 mg |
| | Benzyl alcohol | 40 mg |
| | | 2000 mg |

The obtained ointment was observed under a microscope, and as a result, the preparation was not found to show precipitation of Compound (I), and maintained the homogeneity in the preparation.

### EXAMPLE 6

An external preparation including the following composition was prepared according to the conventional manner. Isopropyl myristate (solubility of Compound (I): about 10 mg/mL) (40 mg) was added to the white Vaseline (1954 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 12

| | | |
|---|---|---|
| Prescription | Compound (I) | 6 mg |
| | White vaseline | 1954 mg |
| | Isopropyl myristate | 40 mg |
| | | 2000 mg |

The obtained ointment was observed under a microscope, and as a result, the preparation was not found to show precipitation of Compound (I), and maintained the homogeneity in the preparation.

### EXAMPLE 7

An external preparation including the following composition was prepared according to the conventional manner. Isopropyl myristate (solubility of Compound (I): about 10 mg/mL) (100 mg) was added to the white Vaseline (1894 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 13

| | | |
|---|---|---|
| Prescription | Compound (I) | 6 mg |
| | White vaseline | 1894 mg |
| | Isopropyl myristate | 100 mg |
| | | 2000 mg |

The obtained ointment was observed under a microscope, and as a result, the preparation was not found to show precipitation of Compound (I), and maintained the homogeneity in the preparation.

### EXAMPLE 8

An external preparation including the following composition was prepared according to the conventional manner. Bleached beeswax (160 mg), liquid paraffin (240 mg), propylene carbonate (solubility of Compound (I): about 33 mg/mL) (40 mg) and isopropyl myristate (solubility of Compound (I): about 10 mg/mL) (40 mg) were added to the white Vaseline (1514 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 14

| | | | |
|---|---|---|---|
| Prescription | Compound (I) | 6 | mg |
| | White vaseline | 1514 | mg |
| | Bleached beeswax | 160 | mg |
| | Liquid paraffin | 240 | mg |
| | Propylene carbonate | 40 | mg |
| | Isopropyl myristate | 40 | mg |
| | | 2000 | mg |

The obtained ointment was observed under a microscope, and as a result, the preparation was not found to show precipitation of Compound (I), and maintained the homogeneity in the preparation.

### EXAMPLE 9

An external preparation including the following composition was prepared according to the conventional manner. Bleached beeswax (160 mg), liquid paraffin (240 mg), propylene carbonate (solubility of Compound (I): about 33 mg/mL) (40 mg) and isopropyl myristate (solubility of Compound (I): about 10 mg/mL) (100 mg) were added to the white Vaseline (1454 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 15

| | | | |
|---|---|---|---|
| Prescription | Compound (I) | 6 | mg |
| | White vaseline | 1454 | mg |
| | Bleached beeswax | 160 | mg |
| | Liquid paraffin | 240 | mg |
| | Propylene carbonate | 40 | mg |
| | Isopropyl myristate | 100 | mg |
| | | 2000 | mg |

The obtained ointment was observed under a microscope, and as a result, the preparation was not found to show precipitation of Compound (I), and maintained the homogeneity in the preparation.

### EXAMPLE 10

An external preparation including the following composition was prepared according to the conventional manner. Bleached beeswax (160 mg), liquid paraffin (240 mg), propylene carbonate (solubility of Compound (I): about 33 mg/mL) (100 mg) and isopropyl myristate (solubility of Compound (I): about 10 mg/mL) (40 mg) were added to the white Vaseline (1454 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 16

| | | | |
|---|---|---|---|
| Prescription | Compound (I) | 6 | mg |
| | White vaseline | 1454 | mg |
| | Bleached beeswax | 160 | mg |
| | Liquid paraffin | 240 | mg |
| | Propylene carbonate | 100 | mg |
| | Isopropyl myristate | 40 | mg |
| | | 2000 | mg |

The obtained ointment was observed under a microscope, and as a result, the preparation was not found to show precipitation of Compound (I), and maintained the homogeneity in the preparation.

### EXAMPLE 11

An external preparation including the following composition was prepared according to the conventional manner. Bleached beeswax (160 mg), liquid paraffin (240 mg), propylene carbonate (solubility of Compound (I): about 33 mg/mL) (100 mg) and isopropyl myristate (solubility of Compound (I): about 10 mg/mL) (40 mg) were added to the white Vaseline (1440 mg) with heating and stirring, and dispersed therein. Compound (I) (20 mg) was added to the dispersion, and the resulting mixture was heated with continuously stirring. Subsequently, the mixture was left to stand at room temperature (about 25° C.) to obtain the external ointment.

TABLE 17

| | | | |
|---|---|---|---|
| Prescription | Compound (I) | 20 | mg |
| | White vaseline | 1440 | mg |
| | Bleached beeswax | 160 | mg |
| | Liquid paraffin | 240 | mg |
| | Propylene carbonate | 100 | mg |
| | Isopropyl myristate | 40 | mg |
| | | 2000 | mg |

The obtained ointment was observed under a microscope, and as a result, the preparation was not found to show precipitation of Compound (I), and maintained the homogeneity in the preparation.

### EXAMPLE 12

An external preparation including the following composition was prepared according to the conventional manner. Bleached beeswax (160 mg), liquid paraffin (180 mg), propylene carbonate (solubility of Compound (I): about 33 mg/mL) (100 mg) and isopropyl myristate (solubility of Compound (I): about 10 mg/mL) (100 mg) were added to the white Vaseline (1454 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 18

| | | | |
|---|---|---|---|
| Prescription | Compound (I) | 6 | mg |
| | White vaseline | 1454 | mg |
| | Bleached beeswax | 160 | mg |
| | Liquid paraffin | 180 | mg |
| | Propylene carbonate | 100 | mg |
| | Isopropyl myristate | 100 | mg |
| | | 2000 | mg |

The obtained ointment was observed under a microscope, and as a result, the preparation was not found to show precipitation of Compound (I), and maintained the homogeneity in the preparation.

### EXAMPLE 13

An external preparation including the following composition was prepared according to the conventional manner. Bleached beeswax (160 mg), liquid paraffin (180 mg), propylene carbonate (solubility of Compound (I): about 33 mg/mL) (100 mg) and isopropyl myristate (solubility of Compound (I): about 10 mg/mL) (100 mg) were added to the white Vaseline (1440 mg) with heating and stirring, and dispersed therein. Compound (I) (20 mg) was added to the dispersion, and the resulting mixture was heated with continuously stirring. Subsequently, the mixture was left to stand at room temperature (about 25° C.) to obtain the external ointment.

TABLE 19

| | | | |
|---|---|---|---|
| Prescription | Compound (I) | 20 | mg |
| | White vaseline | 1440 | mg |
| | Bleached beeswax | 160 | mg |
| | Liquid paraffin | 180 | mg |
| | Propylene carbonate | 100 | mg |
| | Isopropyl myristate | 100 | mg |
| | | 2000 | mg |

The obtained ointment was observed under a microscope, and as a result, the preparation was not found to show precipitation of Compound (I), and maintained the homogeneity in the preparation.

### EXAMPLE 14

An external preparation including the following composition was prepared according to the conventional manner. Bleached beeswax (160 mg), liquid paraffin (320 mg), benzyl alcohol (solubility of Compound (I): about 100 mg/mL) (20 mg) and isopropyl myristate (solubility of Compound (I): about 10 mg/mL) (40 mg) were added to the white Vaseline (1454 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 20

| | | | |
|---|---|---|---|
| Prescription | Compound (I) | 6 | mg |
| | White vaseline | 1454 | mg |
| | Bleached beeswax | 160 | mg |
| | Liquid paraffin | 320 | mg |
| | Benzyl alcohol | 20 | mg |
| | Isopropyl myristate | 40 | mg |
| | | 2000 | mg |

The obtained ointment was observed under a microscope, and as a result, the preparation was not found to show precipitation of Compound (I), and maintained the homogeneity in the preparation.

### EXAMPLE 15

An external preparation including the following composition was prepared according to the conventional manner. Bleached beeswax (160 mg), liquid paraffin (320 mg), benzyl alcohol (solubility of Compound (I): about 100 mg/mL) (20 mg) and isopropyl myristate (solubility of Compound (I): about 10 mg/mL) (40 mg) were added to the white Vaseline (1440 mg) with heating and stirring, and dispersed therein. Compound (I) (20 mg) was added to the dispersion, and the resulting mixture was heated with continuously stirring. Subsequently, the mixture was left to stand at room temperature (about 25° C.) to obtain the external ointment.

TABLE 21

| | | | |
|---|---|---|---|
| Prescription | Compound (I) | 20 | mg |
| | White vaseline | 1440 | mg |
| | Bleached beeswax | 160 | mg |
| | Liquid paraffin | 320 | mg |
| | Benzyl alcohol | 20 | mg |
| | Isopropyl myristate | 40 | mg |
| | | 2000 | mg |

The obtained ointment was observed under a microscope, and as a result, the preparation was not found to show precipitation of Compound (I), and maintained the homogeneity in the preparation.

### EXAMPLE 16

An external preparation including the following composition was prepared according to the conventional manner. Bleached beeswax (160 mg), liquid paraffin (260 mg), benzyl alcohol (solubility of Compound (I): about 100 mg/mL) (20 mg) and isopropyl myristate (solubility of Compound (I): about 10 mg/mL) (100 mg) were added to the white Vaseline (1454 mg) with heating and stirring, and dispersed therein. Compound (I) (6 mg) was added to the dispersion, and the resulting mixture was heated with continuously stirring. Subsequently, the mixture was left to stand at room temperature (about 25° C.) to obtain the external ointment.

TABLE 22

| | | | |
|---|---|---|---|
| Prescription | Compound (I) | 6 | mg |
| | White vaseline | 1454 | mg |
| | Bleached beeswax | 160 | mg |
| | Liquid paraffin | 260 | mg |
| | Benzyl alcohol | 20 | mg |
| | Isopropyl myristate | 100 | mg |
| | | 2000 | mg |

The obtained ointment was observed under a microscope, and as a result, the preparation was not found to show precipitation of Compound (I), and maintained the homogeneity in the preparation.

### EXAMPLE 17

An external preparation including the following composition was prepared according to the conventional manner. Bleached beeswax (160 mg), liquid paraffin (260 mg), benzyl alcohol (solubility of Compound (I): about 100 mg/mL) (20 mg) and isopropyl myristate (solubility of Compound (I): about 10 mg/mL) (100 mg) were added to the white Vaseline (1440 mg) with heating and stirring, and dispersed therein. Compound (I) (20 mg) was added to the dispersion, and the resulting mixture was heated with continuously stirring. Subsequently, the mixture was left to stand at room temperature (about 25° C.) to obtain the external ointment.

TABLE 23

| | | | |
|---|---|---|---|
| Prescription | Compound (I) | 20 | mg |
| | White vaseline | 1440 | mg |
| | Bleached beeswax | 160 | mg |
| | Liquid paraffin | 260 | mg |
| | Propylene carbonate | 20 | mg |
| | Isopropyl myristate | 100 | mg |
| | | 2000 | mg |

The obtained ointment was observed under a microscope, and as a result, the preparation was not found to show precipitation of Compound (I), and maintained the homogeneity in the preparation.

### EXAMPLE 18

An external preparation including the following composition was prepared according to the conventional manner. Isopropyl myristate (solubility of Compound (I): about 10 mg/mL) (100 mg) was added to the white Vaseline (1877.6 mg) with heating and stirring, and dispersed therein. Compound (I) (20 mg) and betamethasone valerate (2.4 mg) were added to the dispersion, and the resulting mixture was heated with continuously stirring. Subsequently, the mixture was left to stand at room temperature (about 25° C.) to obtain the external ointment.

TABLE 24

| | | | |
|---|---|---|---|
| Prescription | Compound (I) | 20 | mg |
| | Betamethasone valerate | 2.4 | mg |
| | White vaseline | 1877.6 | mg |
| | Isopropyl myristate | 100 | mg |
| | | 2000 | mg |

### EXAMPLE 19

An external preparation including the following composition is prepared according to the conventional manner. Isopropyl myristate (solubility of Compound (I): about 10 mg/mL) (100 mg) is added to the white Vaseline (1878 mg) with heating and stirring, and dispersed therein. Compound (I) (20 mg) and tacrolimus (2 mg) are added to the dispersion, and the resulting mixture is heated with continuously stirring. Subsequently, the mixture is left to stand at room temperature (about 25° C.) to obtain the external ointment.

TABLE 25

| | | | |
|---|---|---|---|
| Prescription | Compound (I) | 20 | mg |
| | Tacrolimus | 2 | mg |
| | White vaseline | 1878 | mg |
| | Isopropyl myristate | 100 | mg |
| | | 2000 | mg |

### EXAMPLE 20

An external preparation including the following composition is prepared according to the conventional manner. Isopropyl myristate (solubility of Compound (I): about 10 mg/mL) (100 mg) is added to the white Vaseline (1879.98 mg) with heating and stirring, and dispersed therein. Compound (I) (10 mg) and tacalcitol (0.02 mg) are added to the dispersion, and the resulting mixture is heated with continuously stirring. Subsequently, the mixture is left to stand at room temperature (about 25° C.) to obtain the external ointment.

TABLE 26

| | | | |
|---|---|---|---|
| Prescription | Compound (I) | 20 | mg |
| | Tacalcitol | 0.02 | mg |
| | White vaseline | 1879.98 | mg |
| | Isopropyl myristate | 100 | mg |
| | | 2000 | mg |

### TEST EXAMPLE 1

### Release Test

Synthetic films made of polycarbonate on which 10 mg each of the external preparations obtained in Example 1 and Comparative Example 2 were uniformly applied, were mounted on a Franz cell (diffusion area 0.5 cm²) maintained at 34° C. 50 parts of an aqueous solution of Macrogol was placed as a receptor liquid, and the aqueous solution was sampled over time. The amounts of the drug released from the external preparations were measured by high performance liquid chromatography.

The results are shown in Table 27.

TABLE 27

Testing composition

| | |
|---|---|
| | Comparative |
| Example 1 | Example 2 |

Drug concentration
[%]

| | |
|---|---|
| 0.3 | 0.3 |

| | |
|---|---|
| | Cumulative amount of |
| Time lapse [hr] | drug released [μg/mL] |

| | | |
|---|---|---|
| 0 | 0 | 0 |
| 1 | 12.6 | 3.56 |
| 2 | 23.1 | 7.07 |
| 4 | 35.8 | 11.5 |
| 6 | 27.5 | 16.5 |
| 8 | 49.3 | 20.3 |
| 10 | 34.6 | 23.1 |
| 12 | 59.0 | 25.1 |

It was shown that the release rate of the active ingredient (Compound (I)) from external preparation obtained in Example 1 was higher than that in Comparative Example 2. That is, the external preparation containing 2% by mass of a solvent component in which the solubility of Compound (I) was 4 mg/mL or more (propylene carbonate), exhibited good release of the active ingredient.

### TEST EXAMPLE 2

### Inhibitory Effect on Oxazolone-Induced Auricular Edema Reaction in Mice

Six-week old BALB/c mice (male, supplied by Charles River Japan, Inc.) were purchased and used for the experiment. After medical inspection and acclimatization of at least one week, 7-week old individuals which showed smooth weight increase and which were not recognized to have any abnormality in the appearance were used for the test. The animals were accommodated in plastic cages in groups of 6 individuals each in a breeding room under the conditions of a temperature of 19 to 25° C. and a humidity of 30 to 70%, under illumination of 12 hours a day (7:00 AM to 7:00 PM), and were bred by allowing them to freely ingest commercially available solid feedstuff and water.

As an antigen solution, oxazolone (Sigma-Aldrich Corp.) was dissolved in acetone (Kanto Chemical Co., Inc.) to prepare a 0.5 w/v % oxazolone-acetone solution. The antigen solution (100 μL) was applied to shaved abdomen of the BALB/c mice to sensitize. The shaving of the abdomen was performed the day before the sensitization. On the fifth day after sensitization, the antigen solution (10 μL) was applied to the inner side of the auricle to induce a reaction.

The various external preparations obtained in the Examples and Comparative Examples were respectively administered by applying on the inner side and outer side of the auricle, in an amount of 10 mg each on each side, that is, 20 mg in total, before 3 hours as well as after 2 hours from the induction of the reaction. This group was called test external preparation administering group. Furthermore, a group subjected to sensitization, induction of a reaction, and administration by applying with a placebo external preparation before 3 hours and after 2 hours from the induction of the reaction, was called Positive control group, while a group subjected to induction of the reaction, without sensitization, and administration by applying with a placebo external preparation containing no Compound (I) before 3 hours and after 2 hours from the induction of the reaction, was called Negative control group. Immediately before and after 24 hours of the induction of the reaction by applying the external preparations, the thicknesses of the auricle in each mouse were measured using Dial Thickness Gauge (Ozaki MFG. CO., Ltd.), and the difference in the thicknesses was taken as the auricular edema. The inhibitory rate (%) of auricular edema was calculated as follows.

Inhibitory rate (%)={(Value obtained from Positive control group)−(Value obtained from Test external preparation administering group)}/{(Value obtained from Positive control group)−(Value obtained from Negative control group)}×100 [Equation 1]

The results are shown in Table 28.

TABLE 28

| Test | |
|---|---|
| external | |
| preparation | Inhibitory rate [%] |
| Example 1 | 19 |
| Example 11 | 30 |
| Example 13 | 35 |
| Comparative | Unmeasurable: Erythema and swelling was |
| Example 2 | confirmed in negative control group |
| Comparative | Unmeasurable: Erythema and swelling was |
| Example 3 | confirmed in negative control group |
| Comparative | Unmeasurable: Erythema and swelling was |
| Example 4 | confirmed in negative control group |
| Comparative | Unmeasurable: Erythema and swelling was |
| Example 5 | confirmed in negative control group |

With regard to the external preparations comprising Compound (I), the external preparations containing 0.5 to 15% by mass of solvent component in which the solubility of Compound (I) was 4 mg/mL or more (Examples 1, 11 and 13) exhibited inhibitory effects on the oxazolone-induced auricular edema reaction. On the other hand, the external preparations containing 20% by mass or more of solvent components in which the solubility of Compound (I) was 4 mg/mL or more (Comparative Examples 2 to 5) were found to be inadequate as external preparations because of their strong influence by the solvent components. Therefore, the external preparation of the present invention which contains Compound (I) and 0.5 to 15% by mass of a solvent component in which the solubility of Compound (I) is 4 mg/mL or more, was found to be effective as a therapeutic and/or preventive agent for chronic skin diseases.

From the results of the microscopic observation in Comparative Examples 1 and 6, and from the results of Test Examples 1 and 2, with regard to an external preparation comprising Compound (I) as an active ingredient, it was found that the preparation containing a solvent component having a solubility of Compound (I) of 4 mg/mL or more in an amount of 0.5 to 15% by mass exhibits excellent efficacy because the preparation contains the active ingredient homogeneously and has good capability for drug release.

### INDUSTRIAL APPLICABILITY

According to the present invention, there can be provided an external preparation comprising 7-[2-(3,5-dichloro-4-pyridyl)-1-oxoethyl]-4-methoxy-spiro[1,3-benzodioxol-2,1′-cyclopentane] or a pharmaceutically acceptable salt thereof, which is effective for various diseases caused by PDE-IV hyperfunction [for example, a chronic skin disease (for example, contact dermatitis, atopic dermatitis, seborrheic dermatitis, nummular eczema, Lichen simplex chronicus Vidal, autosensitive dermatitis, stasis dermatitis, asteatotic eczema, psoriasis, etc.), asthma, COPD, allergic diseases, etc.], as an active ingredient, which has excellent homogeneity and excellent releasability of the active ingredient.

1. An external preparation which comprises 7-[2-(3,5-dichloro-4-pyridyl)-1-oxoethyl]-4-methoxy-spiro[1,3-benzodioxol-2,1′-cyclopentane] represented by Formula (I): 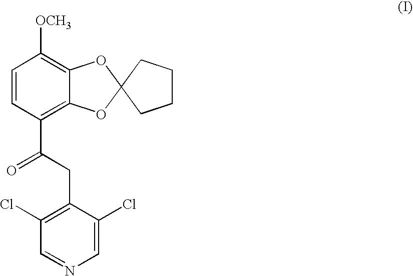
or a pharmaceutically acceptable salt thereof as an active ingredient, comprising 0.5 to 15% by mass of a solvent component in which the solubility of the active ingredient is 4 mg/mL or more.

2. The external preparation according to claim 1, wherein the solvent component is a solvent component in which the solubility of the active ingredient is 5 mg/mL or more.

3. The external preparation according to claim 1 or 2, wherein the solvent component is a solvent component selected from the group consisting of propylene carbonate, dipropylene glycol, isopropyl myristate, isopropyl palmitate, diisopropyl sebacate, diethyl sebacate, benzyl alcohol, ethanol and crotamiton.

4. The external preparation according to claim 1 or 2, wherein the solvent component is propylene carbonate, isopropyl myristate, or a mixed solvent of propylene carbonate and isopropyl myristate.

5. The external preparation according to claim 4, wherein the solvent component is contained in an amount of 2 to 10% by mass.

6. The external preparation according to claim 5, wherein the active ingredient is contained in an amount of 0.1 to 3% by mass.

7. The external preparation according to claim 6, which further comprises a steroid agent.

8. The external preparation according to claim 7, wherein the steroid agent is a compound selected from the group consisting of clobetasol propionate, diflorasone acetate, betamethasone butyrate propionate, mometasone furancarboxylate, difluprednate, dexamethasone propionate, dexamethasone dipropionate, diflucortolone valerate, fluocinonide, amcinonide, halcinonide, hydrocortisone butyrate propionate, deprodone propionate, dexamethasone valerate, prednisolone valerate acetate, fluocinolone acetonide, hydrocortisone butyrate, alclometasone propionate, triamcinolone acetonide, flumethasone pivalate, clobetasone butyrate, hydrocortisone acetate and prednisolone, or a pharmaceutically acceptable salt thereof.

9. The external preparation according to claim 8, which is a therapeutic and/or preventive agent for a disease caused by phosphodiesterase IV hyperfunction.

10. The external preparation according to claim 9, wherein the disease caused by phosphodiesterase IV hyperfunction is a chronic skin disease.

11. The external preparation according to claim 10, wherein the chronic skin disease is a disease selected from the group consisting of contact dermatitis, atopic dermatitis, seborrheic dermatitis, nummular eczema, Lichen simplex chronicus Vidal, autosensitive dermatitis, stasis dermatitis, asteatotic eczema and psoriasis.

12. A method for homogenizing an active ingredient in an external preparation which comprises 7-[2-(3,5-dichloro-4-pyridyl)-1-oxoethyl]-4-methoxy-spiro[1,3-benzodioxol-2,1′-cyclopentane] represented by Formula (I): 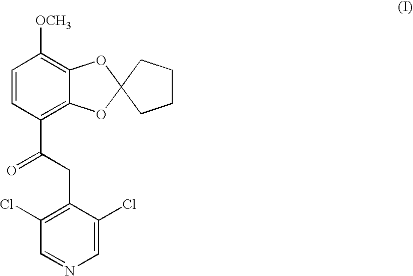
or a pharmaceutically acceptable salt thereof as an active ingredient, comprising allowing a solvent component in which the solubility of the active ingredient is 4 mg/mL or more to exist in the external preparation in an amount of 0.5 to 15% by mass.

13. The method according to claim 12, wherein the solvent component is a solvent component in which the solubility of the active ingredient is 5 mg/mL or more.

14. The method according to claim 12 or 13, wherein the solvent component is a solvent component selected from the group consisting of propylene carbonate, dipropylene glycol, isopropyl myristate, isopropyl palmitate, diisopropyl sebacate, diethyl sebacate, benzyl alcohol, ethanol and crotamiton.

15. The method according to claim 12 or 13, wherein the solvent component is propylene carbonate, isopropyl myristate, or a mixed solvent of propylene carbonate and isopropyl myristate.

16. The method according to claim 14, wherein the solvent component is allowed to exist in the external preparation in an amount of 2 to 10% by mass.

17. The method according to claim 16, wherein the external preparation is an external preparation wherein the active ingredient is contained in an amount of 0.1 to 3% by mass.

18. The method according to claim 17, wherein the external preparation further comprises a steroid agent.

19. The method according to claim 18, wherein the steroid agent is a compound selected from the group consisting of clobetasol propionate, diflorasone acetate, betamethasone butyrate propionate, mometasone furancarboxylate, difluprednate, dexamethasone propionate, dexamethasone dipropionate, diflucortolone valerate, fluocinonide, amcinonide, halcinonide, hydrocortisone butyrate propionate, deprodone propionate, dexamethasone valerate, prednisolone valerate acetate, fluocinolone acetonide, hydrocortisone butyrate, alclometasone propionate, triamcinolone acetonide, flumethasone pivalate, clobetasone butyrate, hydrocortisone acetate and prednisolone, or a pharmaceutically acceptable salt thereof.

20. A method for enhancing the release of an active ingredient from an external preparation which comprises 7-[2-(3,5-dichloro-4-pyridyl)-1-oxoethyl]-4-methoxy-spiro[1,3-benzodioxol-2,1′-cyclopentane] represented by Formula (I): 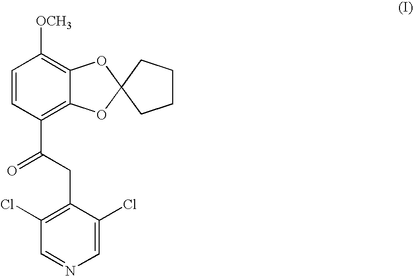
or a pharmaceutically acceptable salt thereof as an active ingredient, comprising allowing a solvent component in which the solubility of the active ingredient is 4 mg/mL or more to exist in the external preparation in an amount of 0.5 to 15% by mass.

21. The method according to claim 20, wherein the solvent component is a solvent component in which the solubility of the active ingredient is 5 mg/mL or more.

22. The method according to claim 20 or 21, wherein the solvent component is a solvent component selected from the group consisting of propylene carbonate, dipropylene glycol, isopropyl myristate, isopropyl palmitate, diisopropyl sebacate, diethyl sebacate, benzyl alcohol, ethanol and crotamiton.

23. The method according to claim 20 or 21, wherein the solvent component is propylene carbonate, isopropyl myristate, or a mixed solvent of propylene carbonate and isopropyl myristate.

24. The method according to claim 22, wherein the solvent component is allowed to exist in the external preparation in an amount of 2 to 10% by mass.

25. The method according to claim 24, wherein the external preparation is an external preparation wherein the active ingredient is contained in an amount of 0.1 to 3% by mass.

26. The method according to claim 25, wherein the external preparation further comprises a steroid agent.

27. The method according to claim 26, wherein the steroid agent is a compound selected from the group consisting of clobetasol propionate, diflorasone acetate, betamethasone butyrate propionate, mometasone furancarboxylate, difluprednate, dexamethasone propionate, dexamethasone dipropionate, diflucortolone valerate, fluocinonide, amcinonide, halcinonide, hydrocortisone butyrate propionate, deprodone propionate, dexamethasone valerate, prednisolone valerate acetate, fluocinolone acetonide, hydrocortisone butyrate, alclometasone propionate, triamcinolone acetonide, flumethasone pivalate, clobetasone butyrate, hydrocortisone acetate and prednisolone, or a pharmaceutically acceptable salt thereof.

28-43. (canceled)
